(19) United States
(12) Patent Application Publication
(10) Pub. No.: US 2020/0345377 A1
(43) Pub. Date: Nov. 5, 2020

(54) Double Embolic Coil Systems and Methods of Manufacture and Use

(71) Applicant: BALT USA, Irvine, CA (US)

(72) Inventors: Jake Le, Foothill Ranch, CA (US); Alois Zauner, Santa Barbara, CA (US)

(21) Appl. No.: 16/862,449

(22) Filed: Apr. 29, 2020

Related U.S. Application Data
(60) Provisional application No. 62/841,116, filed on Apr. 30, 2019.
(60) Provisional application No. 62/844,713, filed on May 7, 2019.

(51) Int. Cl. A61B 17/12

(52) U.S. Cl. CPC A61B 17/12118; A61B 17/12145

(57) ABSTRACT

An embolic coil implant system includes double coils which are made easily assimilated into certain aneurysms and delivered with a profile lower than 0.018 mm, an outer diameter of 0.017 mm.

### CROSS REFERENCE TO RELATED APPLICATIONS

This application claims priority to 62/841,116, filed on Apr. 30, 2019 and 62/844,713, filed on May 7, 2019. The present disclosures relate to the fields of minimally invasive and related neuro-interventional surgery and procedures for making and using novel enhanced devices within the cerebral vasculature of humans and related mammals, and making said devices, expressly incorporated by reference herein are U.S. Pat. Nos. 9,566,071 and 9,566,072 for SYSTEMS AND DEVICES FOR CEREBRAL ANEURYSM REPAIR & COIL SYSTEM, European Patent Number 2996580 commonly owned and assigned and to BALT USA, as if expressly set forth in hac verbae.

### BACKGROUND OF THE DISCLOSURES

U.S. Pat. Nos. 9,566,071 and 9,566,072 are representative of the state of the art. Other patents incorporated by reference as if set forth expressly herein are: U.S. Pat. Nos. 8,066,757; 8,070,791; 8,088,140; 8,197,493; 8,545,514; 8,574,262; 8,585,713 and 9,808,359.

### BRIEF DESCRIPTION OF THE DRAWINGS

FIG. 1 shows the improved double embolic coil of the present invention in a plan view;

FIG. 2 shows a close-up partial perspective view of an exemplary coil implant showing different lengths of the branch coils, according to the present invention; and

FIG. 3 shows positioning of the improved double embolic coil brands within a trunk coil.

### DETAILED DESCRIPTIONS

The present inventors have created a double embolic coil effective for simultaneous delivery of both branches, staggered delivery of each branch and a plurality of diameter combinations.

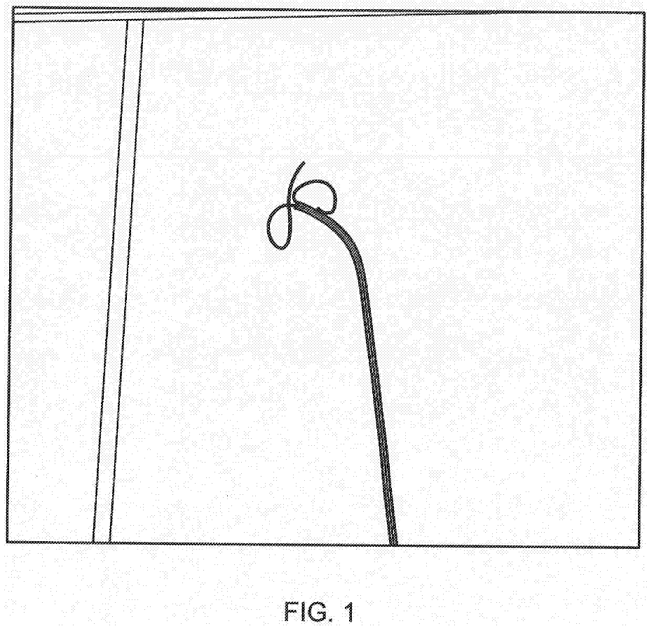

Referring now to FIG. 1, a self-explanatory and illustrative view shows two separate branches of a subject double coil emerging from a trunk coil.

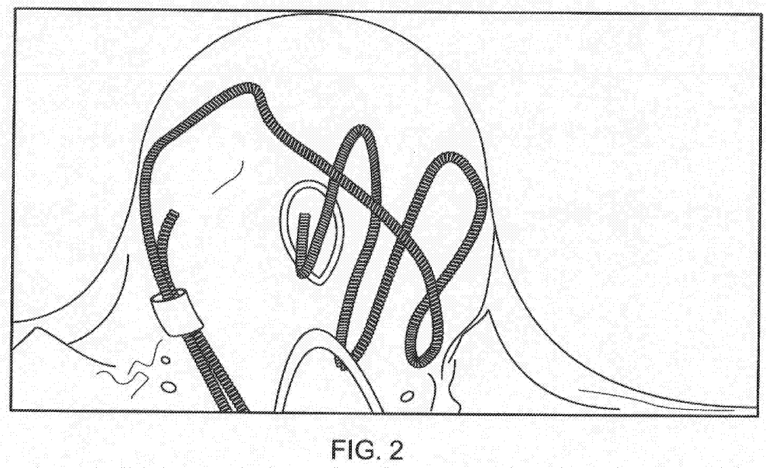

Referring now also to FIG. 2, increased options am available due to the novel structure. According to the present invention, the double coil has a potential improve aneurysm coiling in low aspect ratio aneurysm and wide neck aneurysms. The design of the coil allows each branch of the coil to exit the microcatheter and immediately interact allowing the coil loops to create a more robust frame and decreasing the likelihood of coil loops herniating into the parent artery. Likewise, it has been discovered that having an improved coiling experience in wide neck and low aspect ratio and reduces the need for double catheter technique, balloon assisted coiling, stent assisted coiling. These 3 techniques are used to prevent coil loops herniating into the parent artery.

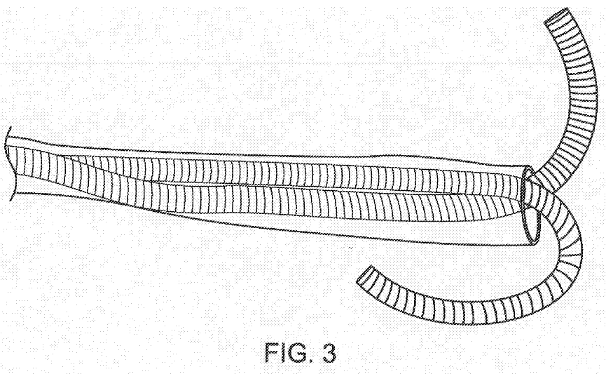

Referring now also to FIG. 3, the flexibility and packageability of double coils is driven by the separate branches angling in several ordinal planes as shown. The present inventors have also done double coil applications in vessel occlusions. Namely, in clinical practice the double coil has claims to improve aneurysm coiling in vessel occlusion procedures. This is because the double coil can simultaneously exit the microcatheter allowing for immediate interaction of the coil loop branches to be able to anchor to the vessel walls, and this simultaneous delivery of coil loop branches may allow for sacrificing shorter vessel lengths, improved stability during deployment and shorter procedure times.

The present inventors have taken existing coil delivery systems European Patent Number 2996580 and engaged and engineered the ability to deliver more than 1 coil, device, stenting means, embolic, tool of any sort, sensor etc., at the same time from a select microcatheter.

Since the prior art does not reveal this innovation it is novel and deserving of letters patent of the United States and such other entities under the Paris Convention and International Law who wish to so recognize it. While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A neurovascular medical device implant system; comprising, in combination:
two separate branches of embolic coils;
affixed to a trunk coil to form double embolic coil implant;
the trunk coil attaches the double embolic coil implant to a delivery pusher; to form a resultory implant assembly;
whereby the entire resultory implant assembly detached as a single unit.

2. A double embolic coil system; comprising, in combination:
two identical coils affixed to a trunk system.

3. The double embolic coil system of claim 2, further comprising two distinct types of coils fixed to a trunk system.

4. The double embolic coil system of claim 2, wherein the subject coils are delivered at the same time.

5. The double embolic coils of claim 4, whereby immediate interaction of each respective coil loop branch is able to anchor to vessel walls.

6. The double embolic coils of claim 5, allowing for sacrificing of shorter vessel lengths, improved stability during deployment and shorter procedure times.

7. The double coil system of claim 6, to improve aneurysm coiling in low aspect ratio aneurysms and wide neck aneurysms.

8. The double coil system of claim 3, to improve aneurysm coiling in low aspect ratio aneurysms and wide neck aneurysms.

9. The double coil system of claim 7, reducing the need for double catheter techniques, balloon assisted coiling and stent assistant coiling.

10. The double coil system of claim 3, reducing the need for means-for-preventing coil loops herniating into a parent artery.

11. The embolic coil system of claim 9, whereby secondary diameters of each branch are at least of one: equal and non-equal diameters;
Complex and helical and one complex and one helical;
Same stiffness or differing stiffnesses;
Primary wind thickness equivalent and different; and
Closed loop and open loop.

12. Methods for making double embolic coil devices, whereby the second branch of the coil is another dence, stenting means or other tool and the profile is around 0.017 mm.

13. The embolic coil system of claim 11, effective to deliver through a small French size of less than 1.7 fr or 0.017 mm for microcatheter outer diameter.
